(19) United States
(12) Patent Application Publication
(10) Pub. No.: US 2006/0155282 A1
(43) Pub. Date: Jul. 13, 2006

(54) Osteosynthesis plate

(76) Inventor: Silvana Vese, Orbassano (Torino) (IT)

Correspondence Address: BACON & THOMAS, PLLC, 625 SLATERS LANE, FOURTH FLOOR, ALEXANDRIA, VA, 22314, US

(21) Appl. No.: 10/546,894

(22) PCT Filed: Jul. 31, 2003
(86) PCT No.: PCT/IB03/03412

§ 371 Date: Aug. 24, 2005

(30) Foreign Application Priority Data
Feb. 28, 2003 (IT) TO2003U000034

(51) Int. Cl. A61F 2/30

(52) U.S. Cl. 606/69

(57) ABSTRACT

An osteosynthesis plate (1) is provided with a plurality of threaded bores (3) in which bushings or barrels (5;13) for the passage of bone screws (7) are screwed; said bone screws (7) being locked in position through threaded plugs (9) that are screwed in the bushings (5) and prevent the unscrewing of said bone screws.

The present invention refers to an osteosynthesis plate, such as for example a femoral plate.

As it is currently known for the repair of bone fractures, particularly femur fractures, there are used suitably-shaped one-piece plates, provided with a plurality of holes or bores to allow the passage of screw to be inserted into the bone.

There are known various types of plates and fastening screws arrangements.

For example WO97/08999 discloses a first type of plate provided with bores for the passage of bone screws in which the heads of the bone screws directly abut against the plate.

A second type of plate provided with barrels housing threaded pins for locking—with respect to the plate—the bone screws previously inserted into the bone is disclosed for example in EP 0649635.

One of the problems encountered when using plates of the above discussed type is the difficulty to firmly secure the bone screw with the plate, once said screw has been fastened in the bone.

A solution to the above problem has been proposed in WO01/91660 that teaches the use of internally hollow bushings screwed in the threaded bores provided in the plate. According to the teaching of this document, there are provided locking bushings with a conical bore the surface of which co-operates with the conical surface of the bone screw passing through the bushing. Alternatively there are provided bushings that are rendered deformable thanks to radial cuts that block the head of the bone screw.

The object of the present invention is to provide an osteosynthesis plate allowing for a reliable and permanent locking of the bone screws passing through such plate, making its manufacturing easier and less expensive, also when the shape of said plate is complex for fitting to several applications.

This and other objects are accomplished through an osteosynthesis plate as claimed in the appended claims.

A main feature of the present invention resides in that it provides removable bushings or barrels that are threaded both on the inner and the outer surface, and provided with closing plugs for locking either the head of the bone screw or the threaded pin engaging the bone screw.

In such a way, the osteosynthesis plate is easy to be made, has a constant thickness for its whole length, thus achieving a notable saving both of working time and of material.

Besides, according to the invention, the bores in the plate can be made inclined or tilted thus allowing an inexpensive manufacturing of particularly shaped plates.

Another advantage resides in that that although the outer diameter of the bushings is fixed for being screwed in the bore of the plate, their inner diameters can be made different in accordance with the size of the bone screw to be fitted inside it.

A not last advantage of the invention is that the bone plate can be used with both bone screws directly screwed into the plate, as well as with bone screws that have been screwed to the plate in advance and are later locked through threaded pins.

The invention will now be disclosed in detail with particular reference to the attached drawings referring to a non limiting embodiment thereof, in which:

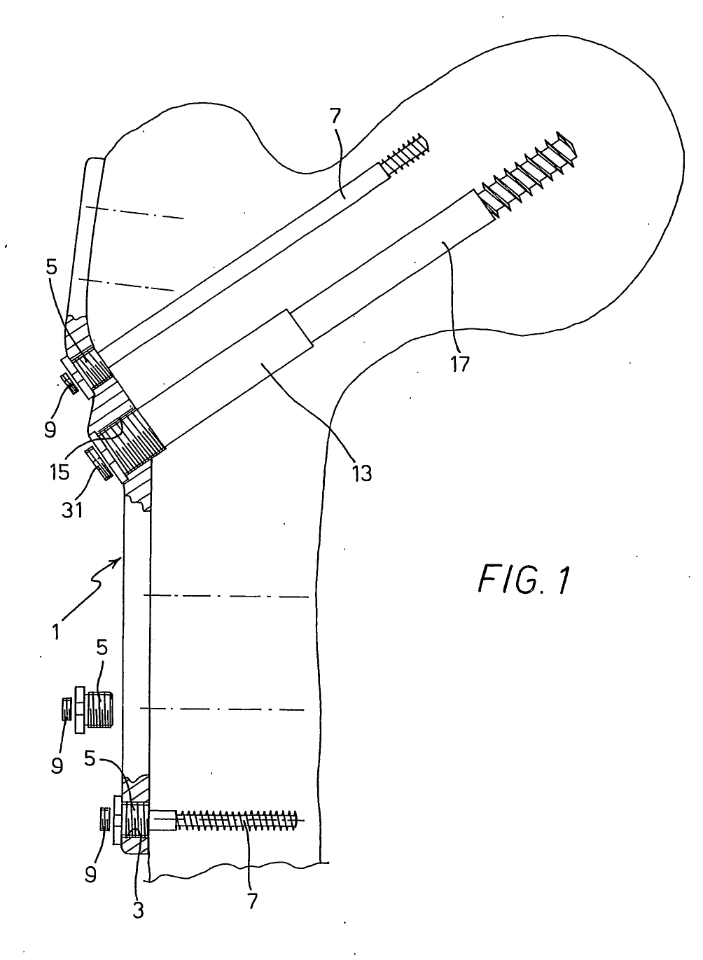

FIG. 1 is a partially cross-sectioned elevation side view of the object osteosynthesis plate;

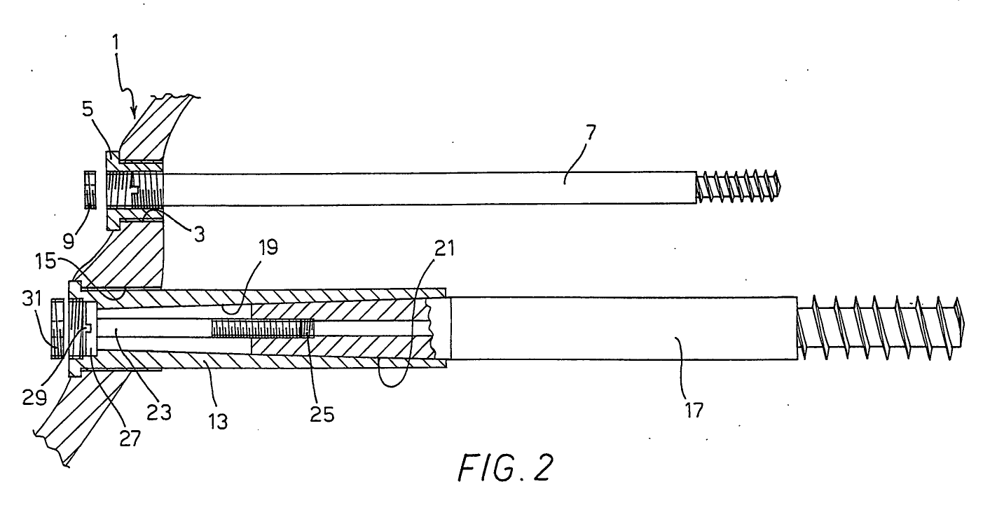

FIG. 2 is a side view of a partial longitudinal cross-section of said osteosynthesis plate;

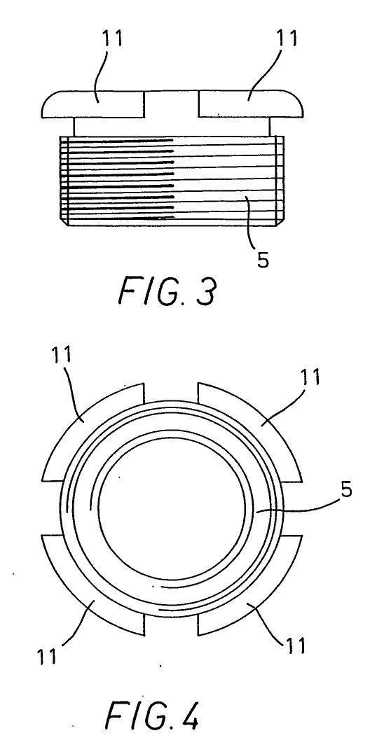

FIG. 3 is a side view of one of said bushings to be fitted to the plate;

FIG. 4 is a plan bottom view of the bushing of FIG. 3.

As clearly shown in the figures, a plate 1 is provided with a plurality of threaded bores 3 in which are screwed as many bushings 5 for the passage of bone screws 7; said bone screws being locked in the desired position through as many threaded plugs 9 that are screwed in the bushings 5 and lock the heads of the screws 7 in said bushings and therefore in the plate 1. Advantageously, the bushing 5 provides for an interrupted radially protruding rim so as to form sectors 11 whereby the bushing can be turned by using a suitable tool.

Preferably the length of the bushing 5 is about equal to the thickness of the plate 1, nevertheless it could have a different length and extend beyond the plate 1 along the bone screw.

The plate 1 further includes a threaded barrel 13 that can be screwed in a bore 15 of the plate 1. Said barrel 13 allows for the passage of a bone screw 17.

According to the invention, the barrel 13 is provided with a slightly (frusto)conical inner bore 19 the cross-section area of which increases when going from the plate towards the outside, towards the bone, and also the (non-threaded) stem of the bone screw 17 comprises a slightly (frusto)conical portion 21 for providing a contact engagement between the two conical parts, i.e. the inner surface 19 of the bore and the stem 21 of the screw.

A (partially) threaded pin or bolt 23 engages an axial bore 25 drilled in the head of the bone screw 17 for strengthening the contact engagement between the conical surfaces 19 and 21 when said screw 17 is turned to be pulled towards the plate 1 when screwing the bolt 23. Also the hollow 29 housing the head 27 of said bolt 23 is closed by a threaded plug 31. The threaded plug 31 allows for locking the bolt 23 and it is similar to the threaded plugs 9 that are screwed in the bushings 5 for locking the screw heads in the plate 1.

1. An osteosynthesis plate comprising a plurality of threaded bores in each of which a bushing or barrel is screwed for the passage of a bone screw; each bone screw being locked in position by a threaded plug that is screwed in a respective bushing or barrel that blocks a respective bone screw in said plate.

2. A plate as claimed in claim 1, wherein said plugs lock the heads of said bone screws in said bushings.

3. A plate as claimed in claim 1, wherein said plugs lock the heads of corresponding threaded pins or bolts engaging the threaded bores provided in the heads of said bone screws in said bushings or barrels.

4. A plate as claimed in claim 2, wherein said bushings or barrels are provided with a (frusto) conical inner surface, the cross-section area of which increases from the plate towards the outside, towards the bone, said inner (frusto) conical surface co-operating with a complementary (frusto) conical outer surface of a bone screw to allow the locking of said screw in said barrel due to the contact engagement between said conical surfaces.

5. A plate as claimed in claim 1, wherein said bushings or barrels comprise interrupted radially protruding rims so as to form sectors whereby said bushing can be turned by means of a suitable tool.

6. A plate as claimed in claim 1, wherein the length of said bushings or barrels is approximately equal to the thickness of said plate.

7. A plate as claimed in claim 1, wherein said bushings or barrels have a length greater than the thickness of the plate and extend for part of the length of bone screws respective that are fitted in said bushings or barrels.

8. A plate as claimed in claim 1, wherein said plate comprises a femoral plate.

9. A plate as claimed in claim 1, wherein said threaded bores are inclined with respect to the plane of said plate.
